(19) United States
(12) Patent Application Publication
(10) Pub. No.: US 2026/0001884 A1
(43) Pub. Date: Jan. 1, 2026

(54) Heterocycle compounds for the treatment of cancer

(71) Applicant: Hoffmann-La Roche Inc., Little Falls, NJ (US)

(72) Inventors: Ming LYU, Shanghai (CN); Hongtao XU, Shanghai (CN); Ge Zou, Shanghai (CN)

(73) Assignee: Hoffmann-La Roche Inc., Little Falls, NJ (US)

(21) Appl. No.: 18/992,101

(22) PCT Filed: Jul. 12, 2023
(86) PCT No.: PCT/EP2023/069251

§ 371 Date: Jan. 7, 2025

(30) Foreign Application Priority Data
Jul. 14, 2022 (WO) PCT/CN2022/105727

(51) Int. Cl. C07D 487/04; A61K 31/513

(52) U.S. Cl. CPC C07D 487/04; A61K 31/513

(57) ABSTRACT

The present invention relates to compounds of formula (I), wherein R¹ to R³, M, A, Y and W are as described herein, and their pharmaceutically acceptable salt thereof, and compositions including the compounds and methods of using the compounds.

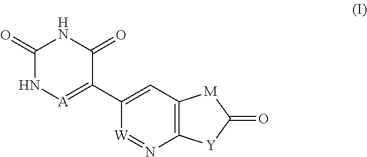

The present invention relates to organic compounds useful for therapy and/or prophylaxis in a mammal, and in particular to inhibition of CD73 useful for treating cancers.

### FIELD OF THE INVENTION

Hyper activation of the adenosine pathway contributes to immunosuppressive tumor microenvironment (TME) that impairs anti-tumor immunity and limits efficacy of immune checkpoint inhibitors. In the last step of the adenosine pathway, the enzyme ecto-5′-nucleotidase (CD73) catalyzes the conversion of AMP to adenosine, which is recognized by the adenosine receptors present in multiple immune cell-types, leading to suppression of the effector T cells and natural killer (NK) cells, activation of the regulatory T (Treg) and myeloid-derived suppressor cells (MDSCs), as well as other changes in the immune system that collectively culminate in an immunosuppressed environment. CD73 is frequently overexpressed in cancers and its upregulation is associated with poor clinical prognosis. Preclinical work in various in vivo tumor models demonstrated restoration of immune cell function and tumor growth inhibition upon genetic ablation or pharmacological inhibition of CD73. Therefore, it is conceivable that alleviating the immunosuppressive TME through CD73 inhibition has the therapeutic potential for restoring anti-tumor immunity and enhancing efficacy of immunotherapy to induce tumor regression. Given the uprising and unmet need for efficacious cancer treatments, inhibition of CD73 activity through administration of small molecule (SM) holds promise. This disclosure describes the invention of new small molecule CD73 inhibitors.

### SUMMARY OF THE INVENTION

The present invention relates to novel compounds of formula (I).

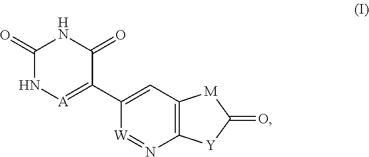

wherein
M is NR¹, wherein R¹ is C₁₋₆alkyl, C₃₋₇cycloalkyl, C₃₋₇cycloalkylC₁₋₆alkyl, or -L¹-R²; wherein L¹ is C₁₋₆alkylene, C₃₋₇cycloalkylene, heterocyclylene or heteroarylene; R² is optionally substituted phenyl, heteroaryl or benzoyl;
Y is NR³, wherein R³ is H, C₁₋₆alkyl, aryl, heteroaryl, C₃₋₇cycloalkyl or C₃₋₇cycloalkylC₁₋₆alkyl;
A is CH or N;
W is CH or N;
or a pharmaceutically acceptable salt thereof.

The compounds of formula (I) show good CD73 inhibition. In another embodiment, the compounds of this invention showed superior cancer cell inhibition. In addition, the compounds of formula (I) also show good or improved human hepatocyte stability, cytotoxicity, solubility profiles.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “halogen” and “halo” are used interchangeably herein and denote fluoro, chloro, bromo, or iodo.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, bicyclo[1.1.1]pentanyl, or bicyclo[2.2.2]octanyl.

The term “C₃₋₇cycloalkylene” denotes a divalent C₃₋₇cycloalkyl group.

The terms “heterocyclic group”, “heterocyclic”, “heterocycle”, “heterocyclyl”, or “heterocyclo” are used interchangeably and refer to any mono-, bi-, tricyclic, spiro or bridged, saturated, partially saturated or unsaturated, non-aromatic ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocycle, regardless of the point of attachment of the cyclic system to the rest of the molecule. In one example, heterocyclyl includes 3-11 ring atoms (“members”) and includes monocycles, bicycles, tricycles, spiro, and bridged ring systems, wherein the ring atoms are carbon, where at least one atom in the ring or ring system is a heteroatom selected from nitrogen, sulfur or oxygen. In other examples, heterocyclyl includes 4-10 or 5-10 ring atoms. In one example, heterocyclyl includes 1 to 4 heteroatoms. In one example, heterocyclyl includes 1 to 3 heteroatoms. In another example, heterocyclyl includes 3- to 7-membered monocycles having 1-2, 1-3 or 1-4 heteroatoms selected from nitrogen, sulfur or oxygen. In another example, heterocyclyl includes 4- to 6-membered monocycles having 1-2, 1-3 or 1-4 heteroatoms selected from nitrogen, sulfur or oxygen. In another example, heterocyclyl includes 3-membered monocycles. In another example, heterocyclyl includes 4-membered monocycles. In another example, heterocyclyl includes 5-6 membered monocycles. In some embodiments, a heterocycloalkyl includes at least one nitrogen. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Examples of heterocycles include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, isoquinolinyl, tetrahydroisoquinolinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, 1,1-dioxoisothiazolyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-onyl, azaspiro[5.5]undecanyl, tetrahydroindolyl, octahydroindolyl, tetrahydroisoindolyl, tetrahydroindazolyl, 1,1-dioxohexahydrothiopyranyl, and 2,3,4a,5,7,7a-hexahydro-[1,4]dioxino[2,3-c]pyrrolyl.

The term “heterocyclylene” denotes a divalent heterocyclyl group.

The term “aryl” denotes a monovalent aromatic carbocyclic mono- or bicyclic ring system comprising 6 to 10 carbon ring atoms. Examples of aryl moieties include phenyl and naphthyl. The term “arylene” denotes a divalent aryl group.

The term “heteroaryl” refers to any mono-, bi-, or tricyclic aromatic ring system containing from 1 to 4 heteroatoms selected from nitrogen, oxygen, and sulfur, and in an example embodiment, at least one heteroatom is nitrogen. See, for example, Lang's Handbook of Chemistry (Dean, J. A., ed.) 13^{th} ed. Table 7-2 [1985]. Included in the definition are any bicyclic groups where any of the above heteroaryl rings are fused to an aryl ring, wherein the aryl ring or the heteroaryl ring is joined to the remainder of the molecule. In one embodiment, heteroaryl includes 5-6 membered monocyclic aromatic groups where one or more ring atoms is nitrogen, sulfur or oxygen. In one embodiment, heteroaryl includes 7-12 membered bicyclic aromatic groups where one or more ring atoms is nitrogen, sulfur or oxygen. Example heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl, 1H-pyrazolo[3,4-d]pyrimidine, 1H-pyrazolo[3,4-d]pyridazine, imidazo[1,5-a]pyrazine, imidazo[5,1-f][1,2,4]triazine, [1.2.4]triazolo[4,3-a]pyrazine, 1H-pyrazolo[3,4-c]pyridazine, 1H-pyrazolo[3,4-b]pyridine, 1H-pyrazolo[4,3-d]pyrimidine, 1H-pyrazolo[3,4-c]pyridine, 1H-pyrazolo[4,3-c]pyridine and purinyl, as well as benzo-fused derivatives, for example benzoxazolyl, benzofuryl, benzothiazolyl, benzothiadiazolyl, benzotriazolyl, benzoimidazolyl, indazolyl and indolyl.

The term “heteroarylene” denotes a divalent heteroaryl group.

In particular embodiments, a heterocyclyl group or a heteroaryl group is attached at a carbon atom of the heterocyclyl group or the heteroaryl group. By way of example, carbon bonded heterocyclyl groups include bonding arrangements at position 2, 3, 4, 5, or 6 of a pyridine ring, position 3, 4, 5, or 6 of a pyridazine ring, position 2, 4, 5, or 6 of a pyrimidine ring, position 2, 3, 5, or 6 of a pyrazine ring, position 2, 3, 4, or 5 of a furan, tetrahydrofuran, thiofuran, thiophene, pyrrole or tetrahydropyrrole ring, position 2, 4, or 5 of an oxazole, imidazole or thiazole ring, position 3, 4, or 5 of an isoxazole, pyrazole, or isothiazole ring, position 2 or 3 of an aziridine ring, position 2, 3, or 4 of an azetidine ring, position 2, 3, 4, 5, 6, 7, or 8 of a quinoline ring or position 1, 3, 4, 5, 6, 7, or 8 of an isoquinoline ring.

In certain embodiments, the heterocyclyl group or heteroaryl group is N-attached. By way of example, nitrogen bonded heterocyclyl or heteroaryl groups include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

In one embodiment, the skilled in the art can understand the keto-enol tautomerism exists for certain structures as illustrated below:

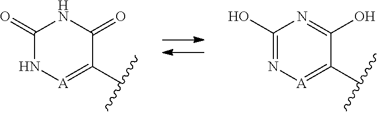

The term “optionally substituted” unless otherwise specified means that a group may be unsubstituted or substituted by one or more (e.g., 0, 1, 2, 3, 4, or 5 or more, or any range derivable therein) of the substituents listed for that group in which said substituents may be the same or different. In an embodiment, an optionally substituted group has 1 substituent. In another embodiment an optionally substituted group has 2 substituents. In another embodiment an optionally substituted group has 3 substituents. In another embodiment an optionally substituted group has 4 substituents. In another embodiment an optionally substituted group has 5 substituents.

The term “protecting group” or “PG” denotes the group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Inhibitor of CD73

The present invention relates to (i) a compound of formula (I),

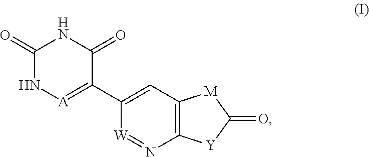

wherein
M is NR₁, wherein R¹ is C₁₋₆alkyl, C₃₋₇cycloalkyl, C₃₋₇cycloalkylC₁₋₆alkyl, or -L¹-R²; wherein L¹ is C₁₋₆alkylene, C₃₋₇cycloalkylene, heterocyclylene or heteroarylene; R² is optionally substituted phenyl, heteroaryl or benzoyl;
Y is NR₃, wherein R³ is H, C₁₋₆alkyl, aryl, heteroaryl, C₃₋₇cycloalkyl or C₃₋₇cycloalkylC₁₋₆alkyl;
A is CH or N;
W is CH or N;
or a pharmaceutically acceptable salt thereof.

A further embodiment of present invention is (ii) a compound of formula (I) according to (i), or a pharmaceutically acceptable salt thereof, wherein C₁₋₆alkyl or -L¹-R²; wherein L¹ is C₁₋₆alkylene; R² is phenyl.

A further embodiment of present invention is (iii) a compound of formula (I) according to (i) or (ii), or a pharmaceutically acceptable salt thereof, wherein R¹ is methyl, isopropyl or benzyl.

A further embodiment of present invention is (iv) a compound of formula (I) according to any one of (i) to (iii), or a pharmaceutically acceptable salt thereof, wherein R³ is H or C₁₋₆alkyl.

A further embodiment of present invention is (v) a compound of formula (I) according to any one of (i) to (iv), wherein R³ is H or methyl.

A further embodiment of present invention is (vi) a compound of formula (I) or a pharmaceutically acceptable salt thereof, according to any one of (i) to (v), wherein A is CH.

A further embodiment of present invention is (vii) a compound of formula (I) or a pharmaceutically acceptable salt thereof, according to any one of (i) to (vi), wherein W is N.

A further embodiment of present invention is (viii) a compound of formula (I), wherein
M is NR¹, wherein R¹ is C₁₋₆alkyl or -L¹-R²; wherein L¹ is C₁₋₆alkylene; R² is phenyl;
Y is NR³, wherein R³ is H or C₁₋₆alkyl;
A is CH;
W is N;
or a pharmaceutically acceptable salt thereof.

A further embodiment of present invention is (ix) a compound of formula (I), wherein
M is NR¹, wherein R¹ is methyl, isopropyl or benzyl;
Y is NR³, wherein R³ is H or methyl;
A is CH;
W is N;
or a pharmaceutically acceptable salt thereof.

Another embodiment of present invention is (x) a compound of formula (I) selected from the following:
5-(5-methyl-6-oxo-7H-imidazo[4,5-c]pyridazin-3-yl)-1H-pyrimidine-2,4-dione;
5-(5,7-dimethyl-6-oxo-imidazo[4,5-c]pyridazin-3-yl)-1H-pyrimidine-2,4-dione;
5-(5-isopropyl-7-methyl-6-oxo-imidazo[4,5-c]pyridazin-3-yl)-1H-pyrimidine-2,4-dione; and
5-(5-benzyl-7-methyl-6-oxo-imidazo[4,5-c]pyridazin-3-yl)-1H-pyrimidine-2,4-dione;
or a pharmaceutically acceptable salt thereof.

Another embodiment of present invention is related to (xi) a process for the preparation of a compound according to any one of (i) to (x) comprising the following step:

a) Deprotection of compound of formula (VII),

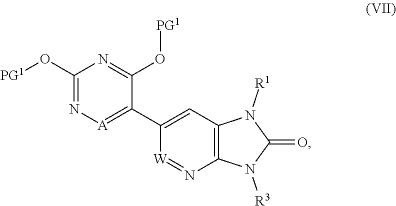

with acid or through hydrogenation to afford the compound of formula (VIII),

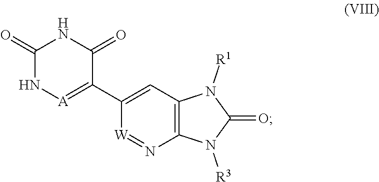

wherein PG¹ is methyl, tert-butyl, TBS, ethoxymethyl, or benzyl. R¹, R³, A and W are defined as in any one of (i) to (ix); the acid in step a) is HCl.

Another embodiment of present invention is (xii) a compound or pharmaceutically acceptable salt according to any one of (i) to (x) for use as therapeutically active substance.

Another embodiment of present invention is (xiii) a pharmaceutical composition comprising a compound in accordance with any one of (i) to (x) and a pharmaceutically acceptable excipient.

Another embodiment of present invention is (xiv) the use of a compound according to any one of (i) to (x) for treating cancers.

Another embodiment of present invention is (xv) the use according to (xiv), wherein the cancer is pancreatic cancer, colorectal cancer, gastric cancer, esophageal cancer, liver cancer, lung cancer, breast cancer, ovarian cancer, prostate cancer or melanoma.

Another embodiment of present invention is (xvi) the use of a compound according to any one of (i) to (x) for inhibiting CD73.

Another embodiment of present invention is (xvii) the use of a compound according to any one of (i) to (x) for the preparation of a medicament for the treatment or prophylaxis of cancers, wherein the cancer is pancreatic cancer, colorectal cancer, gastric cancer, esophageal cancer, head and neck cancer, liver cancer, lung cancer, breast cancer, ovarian cancer, prostate cancer, melanoma, multiple myeloma, acute myeloid leukemia, or acute and chronic lymphoblastic leukemia.

Another embodiment of present invention is (xviii) the use of a compound according to any one of (i) to (x) for the preparation of a medicament as a CD73 inhibitor.

Another embodiment of present invention is (xix) a compound or pharmaceutically acceptable salt according to any one of (i) to (x), when manufactured according to a process of (xi).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit the enzymatic activity of CD73 protein in converting AMP to adenosine. In one example, the pharmaceutically effective amount of the compound of the invention administered parenterally per dose will be in the range of about 0.01 to 100 mg/kg, alternatively about 0.1 to 50 mg/kg of patient body weight per day, with the typical initial range of compound used being 0.3 to 30 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 0.1 mg to 500 mg of the compound of the invention compounded with about 0.1 to 500 mg anhydrous lactose, about 0.1 to 500 mg sodium croscarmellose, about 0.1 to 500 mg polyvinylpyrrolidone (PVP) K30, and about 0.1 to 500 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 1 to 450 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of cancers. Another embodiment includes a pharmaceutical composition comprising a compound of formula (I) for use in the treatment of cancer.

The following embodiments illustrate typical compositions of the present invention, but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention inhibit the enzymatic activity of CD73 in converting AMP to adenosine. Accordingly, the compounds of the invention are useful for reducing the adenosine levels in the TME. Compounds of the invention are useful for promoting immune-mediated killing of cancer cells that overexpress CD73, e.g. pancreatic cancer, colorectal cancer, gastric cancer, esophageal cancer, head and neck cancer, liver cancer, lung cancer, breast cancer, ovarian cancer, prostate cancer, melanoma, multiple myeloma, acute myeloid leukemia, or acute and chronic lymphoblastic leukemia. Alternatively, compounds of the invention are useful for promoting immune-mediated killing of cancer cells that are dependent on the adenosine pathway or in malignant solid tumor where the adenosine pathway is potentiated by dysregulation or mutation of effector pathways as EGFR-RAS-MAPK, PI3K-AKT-driven signaling, for targeted therapy in pancreatic adenocarcinoma, non-small cell lung cancer, esophageal and gastric adenocarcinoma, etc. More broadly, the compounds can be used for the treatment and prophylaxis of all cancer types which exhibit immunosuppressive TME.

Another embodiment includes a method of treating or preventing cancer in a mammal in need of such treatment, wherein the method comprises administering to said mammal a therapeutically effective amount of a compound of formula (I), a stereoisomer, tautomer or pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R³, M, A, Y and W are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

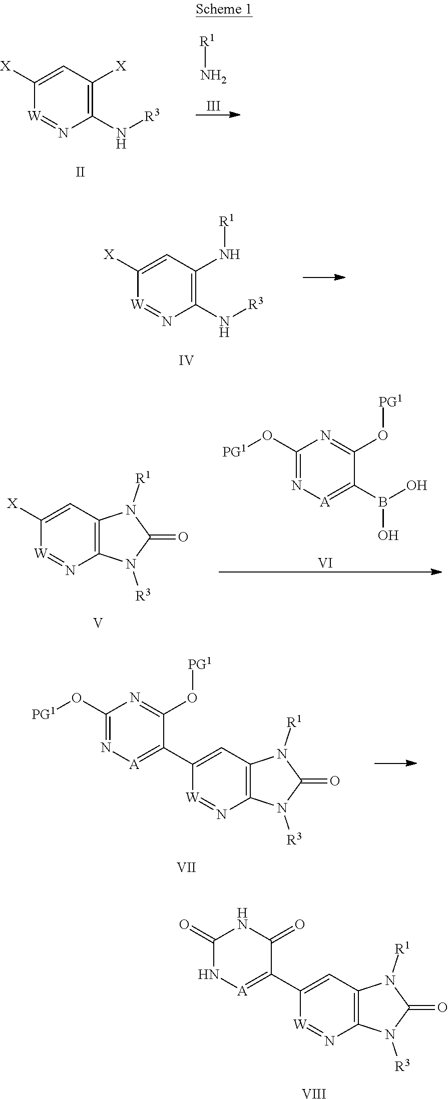

wherein X is halogen; each PG¹ is independently oxygen protecting group (such as methyl, tert-butyl, TBS, ethoxymethyl, or benzyl). Compound of formula (VIII) can be prepared according to Scheme 1. Selective aromatic nucleophilic substitution or metal catalyzed coupling reaction (such as Buchwald-Hartwig amination or Ullmann coupling) of di-halo aniline (II) with amine R¹NH₂ (III) afford compound of formula (IV). The compound of formula (IV) can be converted to compound of formula (V) via condensation reaction with condensing reagent, such as CDI or triphosgene, in the presence of a base, such as DIEA or TEA. The subsequent coupling of halide (V) with boronic acid (VI) can be achieved by Suzuki coupling conditions with a catalyst, such as Pd(dppf)Cl₂ and cataCXium-A-Pd-G3, and a base, such as Cs₂CO₃ and K₃PO₄, to provide the compound of formula (VII). Deprotection of compound of formula (VII) removing PG¹ with acid, such as HCl, or hydrogenation affords the final compound of formula (VIII).

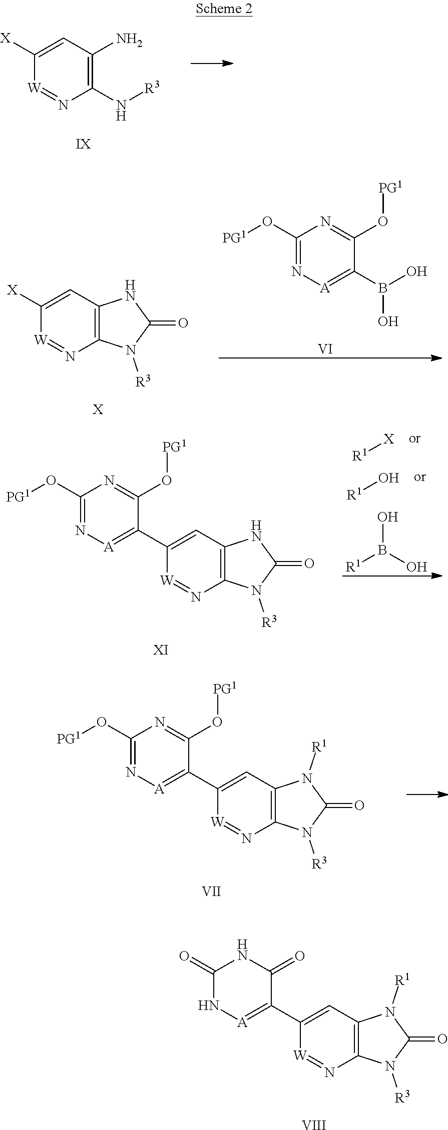

Alternatively, compound of formula (VIII) can be prepared according to Scheme 2. Compound of formula (IX) can be converted to compound of formula (X) via condensation reaction with condensing reagent, such as CDI or triphosgene, in the presence of a base, such as DIEA or TEA. Suzuki coupling of halide (X) with boronic acid (VI) can be achieved in the presence of a catalyst, such as Pd(dppf)Cl₂ and cataCXium-A-Pd-G3, and a base, such as Cs₂CO₃ and K₃PO₄, to provide the compound of formula (XI). Selective N-substitution reaction, or metal catalyzed coupling reaction (such as Buchwald-Hartwig amination, or Ullmann coupling) of compound of formula (XI) with halide R¹X, or Mitsunobu reaction of formula (XI) with alcohol R¹OH, or Chan-Lam coupling of formula (XI) with boronic acid R¹B(OH)₂, can afford compound of formula (VII). Deprotection of compound of formula (VII) removing PG¹ with acid, such as HCl, or hydrogenation affords the final compound of formula (VIII).

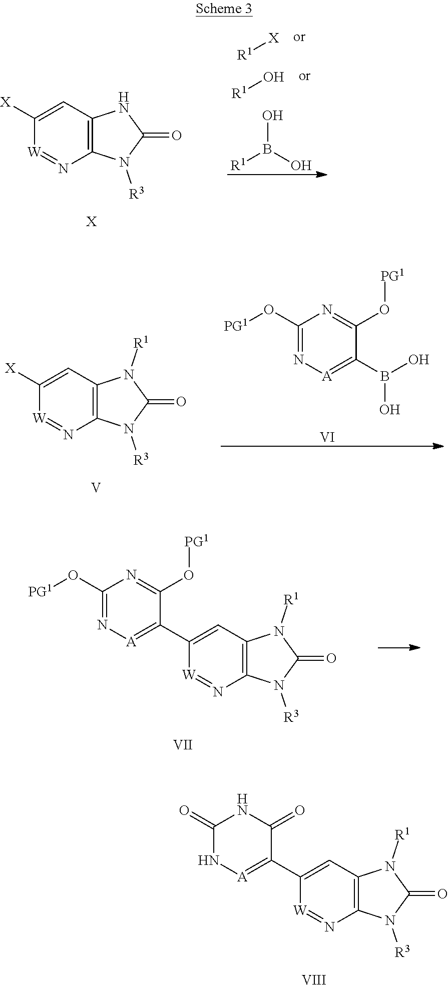

Alternatively, compound of formula (VIII) can be prepared according to Scheme 3. Compound of formula (X) can be converted to compound of formula (V) by selective N-substitution reaction or metal catalyzed coupling reaction (such as Buchwald-Hartwig amination, or Ullmann coupling) of formula (X) with halide RIX, or Mitsunobu reaction of formula (X) with alcohol R¹OH, or Chan-Lam coupling of formula (X) with boronic acid R¹B(OH)₂. The subsequent coupling of halide (V) with boronic acid (VI) can be achieved by Suzuki coupling conditions with a catalyst, such as Pd(dppf)Cl₂ and cataCXium-A-Pd-G3, and a base, such as Cs₂CO₃ and K₃PO₄, to afford the compound of formula (VII). Deprotection of compound of formula (VII) removing PG′ with acid, such as HCl, or hydrogenation affords the final compound of formula (VIII).

Compounds of this invention can be obtained as mixtures of diastereomers or enantiomers, which can be separated by methods well known in the art, e.g. (chiral) HPLC or SFC. In another embodiment, compound of formula (I) can be obtained according to above scheme by using corresponding chiral starting materials.

This invention also relates to a process for the preparation of a compound of formula (I) comprising following step:

a) Deprotection of compound of formula (VII),

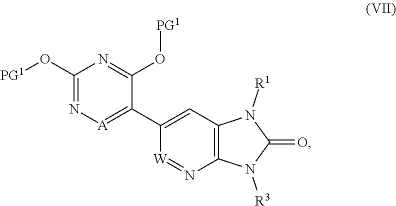

with an acid or through hydrogenation to afford the compound of formula (VIII),

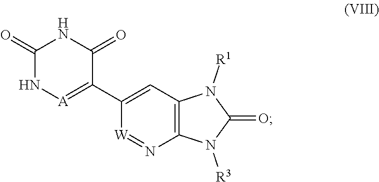

wherein

in step a) the acid can be, for example, HCl.

A compound of formula (I) when manufactured according to the above process is also an object of the invention.

### Examples

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN: acetonitrile
aq. Aqueous
Boc₂O: di-tert-butyl dicarbonate
CataCXium-A-Pd-G3 [(di(1-adamantyl)-butylphosphine)-2-(2′-amino-1,1′-biphenyl)]palladium(II) methanesulfonate
CDI: N,N′-carbonyldiimidazole
DCM: dichloromethane
DIPEA or DIEA: N,N-diisopropylethylamine
DMA: N,N-Dimethylacetamide
Pd(dppf)Cl₂·DCM: [1,1′-Bis(diphenylphosphino)ferrocene]dichloropalladium(II), complex with dichloromethane
EA or EtOAc: ethyl acetate
FA: formic acid
IC₅₀: half inhibition concentration
LCMS: liquid chromatography-mass spectrometry
MS: mass spectrometry
PE: petroleum ether
prep-HPLC: preparative high performance liquid chromatography
prep-TLC: preparative thin layer chromatography
rt: room temperature
RT retention time
SFC: supercritical fluid chromatography
TFA: trifluoroacetic acid
TLC: thin layer chromatography
v/v: volume ratio

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃. H₂O in H₂O; B: acetonitrile;
Basic condition II: A: 0.025% NH₃· H₂O in H₂O; B: acetonitrile;
Neutral condition: A: H₂O; B: acetonitrile.
Mass spectra (MS): generally only ions which indicate the parent mass are reported, and unless otherwise stated the mass ion quoted is the positive mass ion (MH)⁺.

NMR Spectra were obtained using Bruker Avance 400 MHz or 500 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Example 1

### 5-(5-methyl-6-oxo-7H-imidazo[4,5-c]pyridazin-3-yl)-1H-pyrimidine-2,4-dione

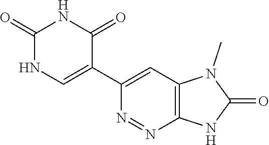 [SMILES: Cn1c(=O)[nH]c2nnc(-c3c[nH]c(=O)[nH]c3=O)cc21]

The titled compound was synthesized according to the following scheme:

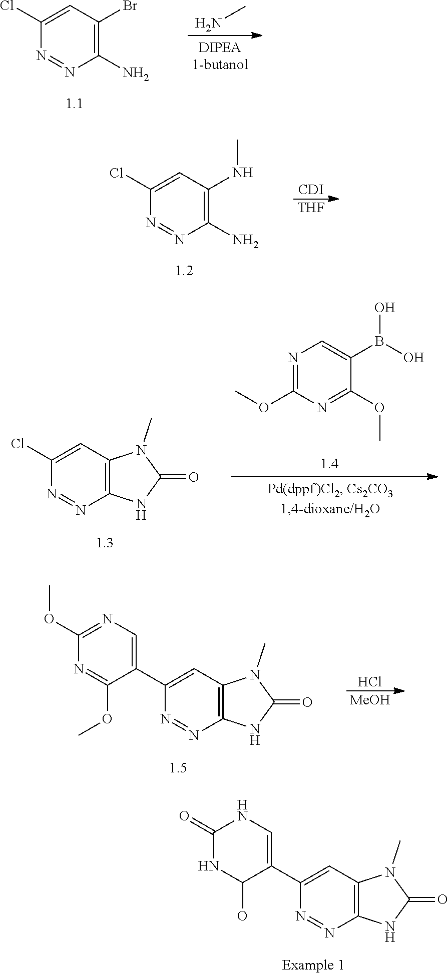

### Step (a): Preparation of 6-chloro-N⁴-methyl-pyridazine-3,4-diamine (compound 1.2)

To a solution of 4-bromo-6-chloro-pyridazin-3-amine (compound 1.1, 5.0 g, 24.0 mmol) in 1-butanol (20 mL) was added DIEA (8.4 mL, 48.0 mmol) and methanamine (2 M in THF, 24.0 mL, 48.0 mmol) under N₂ atmosphere to give a brown solution. The reaction mixture was stirred at 80° C. for 16 hrs, then concentrated and purified by perp-HPLC to afford compound 1.2 (3.5 g). MS: calc'd 159.0 [(M+H)⁺]; measured 159.3 [(M+H)⁺].

### Step (b): Preparation of 3-chloro-5-methyl-7H-imidazo[4,5-c]pyridazin-6-one (compound 1.3)

To a solution of 6-chloro-N⁴-methyl-pyridazine-3,4-diamine (compound 1.2, 200.0 mg, 1.26 mmol) in THF (4 mL) was added CDI (250.0 mg, 1.54 mmol) at room temperature, the reaction mixture was stirred at room temperature for 5 hrs, then concentrated, the resultant residue was purified by prep-HPLC to afford compound 1.3 (80.0 mg). MS: calc'd 185.0 [(M+H)⁺]; measured 185.0 [(M+H)⁺].

### Step (c): Preparation of 3-(2,4-dimethoxypyrimidin-5-yl)-5-methyl-7H-imidazo[4,5-c]pyridazin-6-one (compound 1.5)

To a solution of 3-chloro-5-methyl-7H-imidazo[4,5-c]pyridazin-6-one (compound 1.3, 80.0 mg, 0.43 mmol) in 1,4-dioxane (2 mL) and water (0.4 mL) was added 2,4-dimethoxypyrimidine-5-boronic acid (compound 1.4, 87.7 mg, 0.48 mmol), Cs₂CO₃ (423.6 mg, 1.3 mmol) and Pd(dppf)Cl₂·DCM (35.4 mg, 0.04 mmol) under N₂. The resultant mixture was stirred at 80° C. for 4 hrs, then quenched with water, extracted with EA for three times. The combined organic layer was washed with brine, dried over Na₂SO₄ and concentrated in vacuo. The residue was purified by prep-HPLC to afford compound 1.5 (20.0 mg, 16.0% yield). MS: calc'd 289.1 [(M+H)⁺]; measured 289.1 [(M+H)⁺].

### Step (d): Preparation of 5-(5-methyl-6-oxo-7H-imidazo[4,5-c]pyridazin-3-yl)-1H-pyrimidine-2,4-dione (Example 1)

To a solution of 3-(2,4-dimethoxypyrimidin-5-yl)-5-methyl-7H-imidazo[4,5-c]pyridazin-6-one (compound 1.5, 20.0 mg, 0.07 mmol) in methanol (0.2 mL) was added 2 M HCl (0.5 mL, 1.0 mmol). After being stirred at 60° C. for 1 h, precipitation formed and the solid was collected by filtration, washed by MeOH, and lyophilized to afford Example 1 (2.22 mg). MS: calc'd 261.1 [(M+H)⁺]; measured 261.0 [(M+H)⁺]. ¹H NMR (400 MHZ, DMSO-d₆) δ=12.7 (s, 1H), 12.05-11.92 (m, 1H), 11.78 (br s, 1H), 8.36 (br s, 1H), 8.05 (s, 1H), 3.37 (s, 3H).

### Example 2

### 5-(5,7-dimethyl-6-oxo-imidazo[4,5-c]pyridazin-3-yl)-1H-pyrimidine-2,4-dione

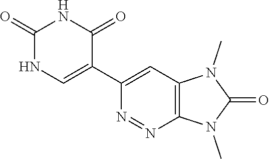 [SMILES: Cn1c(=O)n(C)c2nnc(-c3c[nH]c(=O)[nH]c3=O)cc21]

The titled compound was synthesized according to the following scheme:

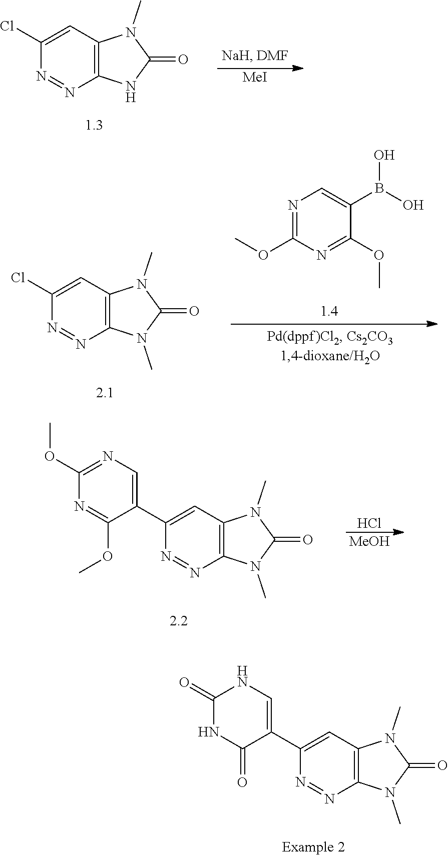

### Step (a): Preparation of 3-chloro-5,7-dimethyl-imidazo[4,5-c]pyridazin-6-one (compound 2.1)

To a solution of 3-chloro-5-methyl-7H-imidazo[4,5-c]pyridazin-6-one (compound 1.3, 300.0 mg, 1.63 mmol) in DMF (3 mL) was added sodium hydride (60% dispersion in mineral oil, 130.0 mg, 3.25 mmol) at 0° C. The reaction mixture was stirred for another 30 min, then iodomethane (346.0 mg, 2.44 mmol) was added at 0° C. The resultant mixture was stirred at room temperature for 1 hr, then quenched with water, extracted with EA for three times. The combined organic layer was washed with brine, dried over Na₂SO₄ and concentrated in vacuo. The residue was purified by silica gel column chromatography (eluent: 0% to 20% MeOH in DCM) to afford compound 2.1 (220.0 mg, 68.2% yield). MS: calc'd 199.0 [(M+H)⁺]; measured 199.0 [(M+H)⁺].

### Step (b): Preparation of 5-(5,7-dimethyl-6-oxo-imidazo[4,5-c]pyridazin-3-yl)-1H-pyrimidine-2,4-dione (Example 2)

5-(5,7-dimethyl-6-oxo-imidazo[4,5-c]pyridazin-3-yl)-1H-pyrimidine-2,4-dione (Example 2) was prepared in analogy to Example 1, by replacing 3-chloro-5-methyl-7H-imidazo[4,5-c]pyridazin-6-one (compound 1.3) with 3-chloro-5,7-dimethyl-imidazo[4,5-c]pyridazin-6-one (compound 2.1) in step (c). 10.6 mg of Example 2 was obtained. MS: calc'd 275.1 [(M+H)⁺]; measured 275.2 [(M+H)⁺]. ¹H NMR (400 MHZ, DMSO-d₆) δ=11.45 (s, 1H), 8.13 (s, 1H), 7.86 (s, 1H), 3.42 (s, 3H), 3.35 (s, 3H)

### Example 3

### 5-(5-isopropyl-7-methyl-6-oxo-imidazo[4.5-]pyridazin-3-yl)-1H-pyrimidine-2,4-dione

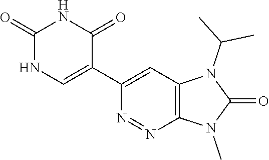 [SMILES: CC(C)n1c(=O)n(C)c2nnc(-c3c[nH]c(=O)[nH]c3=O)cc21]

The titled compound was synthesized according to the following scheme:

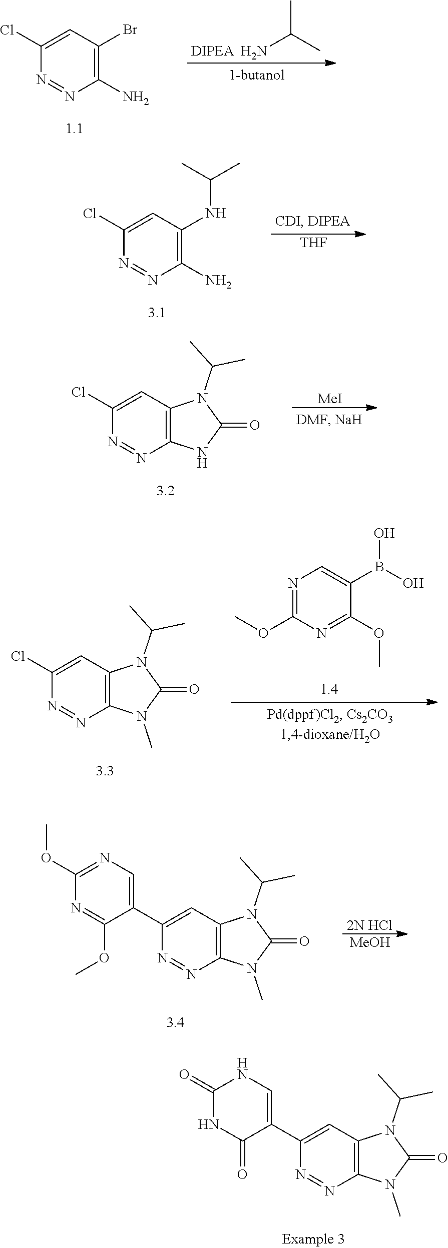

### Step (a): Preparation of 6-chloro-N⁴-isopropyl-pyridazine-3,4-diamine (compound 3.1)

To a solution of 4-bromo-6-chloro-pyridazin-3-amine (compound 1.1, 5.0 g, 23.99 mmol) in n-BuOH (20 mL) was added DIEA (8.4 mL, 47.98 mmol) and isopropylamine (2.41 g, 40.78 mmol) at room temperature. The resultant mixture was stirred at 120° C. for 12 hrs. After being cooled to room temperature, the mixture was concentrated in vacuo. The residue was purified by silica gel column chromatography (eluent: 50% to 100% EA in PE) to afford compound 3.1 (1.2 g). ¹H NMR (400 MHZ, DMSO-d₆) δ=6.33 (s, 1H), 6.10 (s, 2H), 5.97 (br d, J=7.2 Hz, 1H), 3.72-3.61 (m, 1H), 1.17 (d, J=6.4 Hz, 6H).

### Step (b): Preparation of 3-chloro-5-isopropyl-7H-imidazo[4,5-c]pyridazin-6-one (compound 3.2)

To a solution of 6-chloro-N⁴-isopropyl-pyridazine-3,4-diamine (compound 3.1, 0.50 g, 2.68 mmol) in THF (20 mL) was added CDI (531.1 mg. 3.28 mmol) at room temperature. The resultant mixture was stirred at room temperature for 5 hrs, then concentrated in vacuo. The residue was purified by silica gel column chromatography (eluent: 50% to 75% EA in PE) to afford compound 3.2 (400.0 mg, 68.4% yield). MS: calc'd 213.0, 215.0 [M+H]+ measured 213.2, 215.2 [M+H]⁺

### Step (c): Preparation of 3-chloro-5-isopropyl-7-methyl-imidazo[4,5-c]pyridazin-6-one (compound 3.3)

To a solution of 3-chloro-5-isopropyl-7H-imidazo[4,5-c]pyridazin-6-one (compound 3.2, 350.0 mg, 1.65 mmol) in DMF (2 mL) was added sodium hydride (60% dispersion in mineral oil, 131.7 mg. 3.29 mmol) at 0° C. The resultant mixture was stirred at 0° C. for 30 min, then iodomethane (350.4 mg, 2.47 mmol) was added. The reaction mixture was stirred at room temperature for 1 hr, then quenched by slow addition of aq. sat NH₄Cl (20 mL), extracted with EA (20 mL) for three times. The combined organic layer was washed with brine (30 mL), dried over anhydrous sodium sulfate, filtered, and concentrated in vacuo. The residue was purified by prep-TLC (PE/EA=1/1) to afford compound 3.3 (130.0 mg). MS: calc'd 227.1, 229.1 [(M+H)⁺]; measured 227.1, 229.1 [(M+H)⁺].

### Step (d): Preparation of 3-(2,4-dimethoxypyrimidin-5-yl)-5-isopropyl-7-methyl-imidazo[4,5-c]pyridazin-6-one (compound 3.4)

To a solution of 3-chloro-5-isopropyl-7-methyl-imidazo[4,5-c]pyridazin-6-one (compound 3.3, 130.0 mg, 0.57 mmol) in 1,4-dioxane (2 mL) and water (0.4 mL) were added 2,4-dimethoxypyrimidine-5-boronic acid (compound 1.4, 116.06 mg, 0.63 mmol), Cs₂CO₃ (560.6 mg. 1.72 mmol) and Pd(dppf)Cl₂·DCM (46.9 mg, 0.06 mmol) under N₂. The resultant mixture was stirred at 80° C. for 12 hrs. After being cooled to room temperature, the reaction mixture was concentrated in vacuo. The residue was purified by silica gel column chromatography (eluent: 50% to 80% EA in PE) to afford compound 3.4 (180.0 mg). MS: calc'd 331.1 [(M+H)⁺]; measured 331.1 [(M+H)⁺].

### Step (e): Preparation of 5-(5-isopropyl-7-methyl-6-oxo-imidazo[4,5-c]pyridazin-3-yl)-1H-pyrimidine-2,4-dione (Example 3)

To a solution of 3-(2,4-dimethoxypyrimidin-5-yl)-5-isopropyl-7-methyl-imidazo[4,5-c]pyridazin-6-one (compound 3.4, 91.7 mg, 0.28 mmol) in methanol (0.2 mL) was added 2 M HCl (2.0 mL, 4.0 mmol). The resultant mixture was stirred at 60° C. for 1 hr, then aqueous ammonia (1 mL) and water (2 mL) were added. The solid formed was collected by filtration, washed by MeOH, and lyophilized to afford Example 3 (38.16 mg). MS: calc'd 303.1 [(M+H)⁺]; measured 303.1 [(M+H)⁺]. ¹H NMR (400 MHz, DMSO-d₆) δ=11.47 (s, 1H), 8.13 (s, 1H), 7.96 (s, 1H), 4.63-4.56 (m, 1H), 3.41 (s, 3H), 1.44 (d, J=6.8 Hz, 6H).

### Example 4

### 5-(5-benzyl-7-methyl-6-oxo-imidazo[4,5-c]pyridazin-3-yl)-1H-pyrimidine-2,4-dione

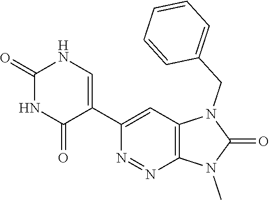 [SMILES: Cn1c(=O)n(Cc2ccccc2)c2cc(-c3c[nH]c(=O)[nH]c3=O)nnc21]

The titled compound was synthesized according to the following scheme:

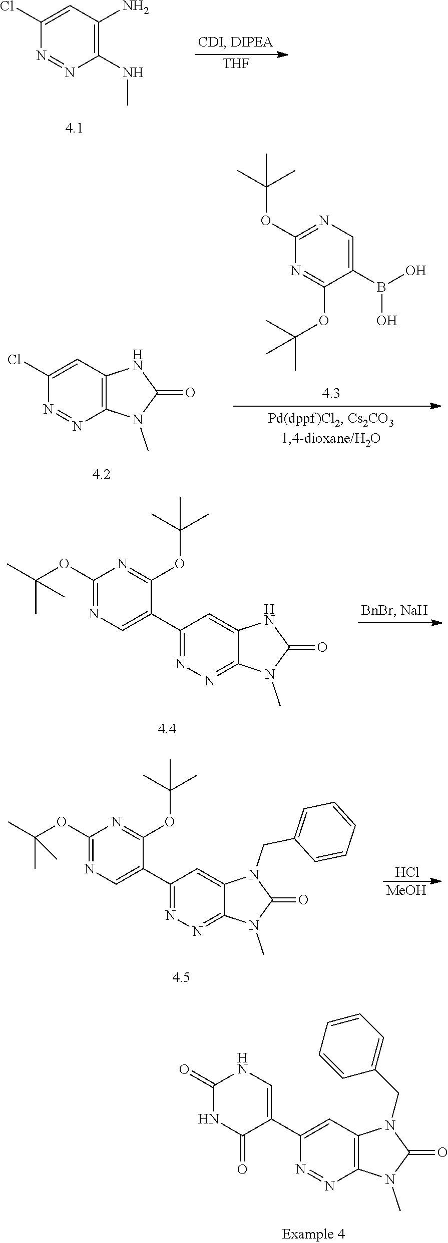

### Step (a): Preparation of 3-chloro-7-methyl-5H-imidazo[4,5-c]pyridazin-6-one (compound 4.2)

To a solution of 6-chloro-N³-methyl-pyridazine-3,4-diamine (compound 4.1, 3.0 g. 18.92 mmol) in THF (30 mL) were added CDI (3.75 g. 23.13 mmol) and DIEA (6.6 mL. 37.83 mmol). The resultant mixture was stirred at 80° C. for 12 hrs. then concentrated in vacuo. The residue was purified by silica gel column chromatography (eluent: 0% to 30% MeOH in DCM) to afford compound 4.2 (2.9 g). MS: calc'd 185.0 [(M+H)⁺]; measured 185.0 [(M+H)⁺].

### Step (b): Preparation of 3-(2,4-ditert-butoxypyrimidin-5-yl)-7-methyl-5H-imidazo[4,5-c]pyridazin-6-one (compound 4.4)

To a solution of (2,4-ditert-butoxypyrimidin-5-yl) boronic acid (compound 4.3, 2.61 g. 9.75 mmol) in 1,4-dioxane (30 mL) and water (5 mL) was added 3-chloro-7-methyl-5H-imidazo[4,5-c]pyridazin-6-one (compound 4.2, 1.5 g. 8.13 mmol). Cs₂CO₃ (7.94 g. 24.38 mmol) and Pd(dppf)Cl₂·DCM (0.66 g. 0.81 mmol) under N₂. The resultant mixture was stirred at 80° C. for 12 hrs. The reaction mixture was concentrated in vacuo. The residue was purified by silica gel column chromatography (eluent: 20% to 100% EA in PE) to afford compound 4.4 (900.0 mg). MS: calc'd 373.2 [(M+H)⁺]; measured 373.1 [(M+H)⁺].

### Step (c): Preparation of 5-benzyl-3-(2,4-ditert-butoxypyrimidin-5-yl)-7-methyl-imidazo[4,5-c]pyridazin-6-one (compound 4.5)

To a solution of 3-(2,4-ditert-butoxypyrimidin-5-yl)-7-methyl-5H-imidazo[4,5-c]pyridazin-6-one (compound 4.4, 100.0 mg, 0.27 mmol) in THF (5 mL) was added NaH (60% dispersion in mineral oil. 21.5 mg, 0.54 mmol) at 0° C. The resultant mixture was stirred at 0° C. for 30 min. then benzyl bromide (0.04 mL. 0.32 mmol) was added. The reaction mixture was stirred at room temperature for 1 hr. then quenched by slow addition of aq. sat NH₄Cl (20 mL), extracted with EA (20 mL) for three times. The combined organic layer was washed with brine (30 mL), dried over anhydrous sodium sulfate, filtered, and concentrated in vacuo. The residue was purified by pre-HPLC to afford compound 4.5 (40.0 mg. 32.2% yield). MS: calc'd 463.2 [(M+H)⁺]; measured 463.2 [(M+H)⁺].

### Step (d): Preparation of 5-(5-benzyl-7-methyl-6-oxo-imidazo[4,5-c]pyridazin-3-yl)-1H-pyrimidine-2,4-dione (Example 4)

To a solution of 5-benzyl-3-(2,4-ditert-butoxypyrimidin-5-yl)-7-methyl-imidazo[4,5-c]pyridazin-6-one (compound 4.5, 40.0 mg, 0.09 mmol) in methanol (0.2 mL) was added 2 M HCl (1.0 mL. 2.0 mmol). The mixture was stirred at room temperature for 1 hr. then aqueous ammonia (1 mL) and water (2 mL) were added to the reaction mixture. The solid formed was collected by filtration, washed by MeOH, and lyophilized to afford Example 4 (15.99 mg). MS: calc'd 351.1 [(M+H)⁺]; measured 351.2 [(M+H)⁺]. ¹H NMR (400 MHZ, DMSO-d₆) ¿=11.56-11.31 (m, 2H), 8.13 (d, J=6.0 Hz, 1H), 7.85 (s, 1H), 7.35-7.28 (m, 5H), 5.10 (s, 2H), 3.46 (s, 3H).

### Biological Example

### Example 5: CD73 Biochemical Assay

Compound serial dilution (1:3) was prepared with Echo 555 liquid handler (Labcyte) into the corresponding wells of a 384-well plate. 25 μL of enzyme working solution [0.2 nM recombinant CD73 protein (purchased from R&D Systems, Inc), 25 mM Tris-HCl pH 7.5, 5 mM MgCl2, 0.01% BSA, 0.01% Brij-35 non-ionic detergent] was added to the assay plate and incubated with compounds for 15 minutes at room temperature. After adding 25 μL of AMP working solution (10 μM AMP, 25 mM Tris-HCl pH 7.5, 5 mM MgCl₂, 0.01% BSA, 0.01% Brij-35) into the assay plate, the reaction was incubated for 10 min at room temperature. 10 uL of Malachite Green A solution was then added to each well for 10 minutes incubation. 10 uL of Malachite B solution was added to each well and then incubated for another 30 minutes. Finally the absorbance value was read on the Envision plate reader (PerkinElmer) at 620 nM. Calculation percentage inhibition by using equation {% inhibition=100×[I−(X−MIN)/(MAX−MIN)]} where X equals to the well signal, Max equals signal of DMSO control and MIN equals signal of no cell control.

TABLE 1
Enzymatic IC₅₀ values of the compounds
of this invention against CD73

| Example No. | IC₅₀ (μM) |
|---|---|
| 1 | 0.622 |
| 2 | 0.254 |
| 3 | 0.267 |

### Example 6: CD73 Cellular Assay

Compound serial dilution (1:3) was prepared with Echo 555 liquid handler (Labcyte) into the corresponding wells of a 384-well plate. 40 μL of MDA-MB-231 cells (ATCC, HTB-26, breast cancer, final concentration at 20,000 cells/mL) suspended in assay buffer (25 mM Tris-HCl pH 7.5, 5 mM MgCl₂, 0.01% BSA, 0.01% Brij-35) were added into the corresponding wells of the plate. After incubating with compounds for 30 minutes, 40 μL of AMP working solution (10 μM AMP, 25 mM Tris-HCl pH 7.5, 5 mM MgCl₂, 0.01% BSA, 0.01% Brij-35) was added into each well in the assay plate. The assay plate was then incubated in a 5% CO₂ incubator (Thermo Fisher Scientific) at 37° C. for 45 min. After the reaction was completed, 50 μL of the supernatants was collected and transferred into a new 384 well plate. 10 μL of Malachite A was added to each well in the assay plate and incubated for 10 minutes. 10u L of Malachite B was then added to each corresponding well of the plate and incubated for 30 minutes. Finally the absorbance value was read on the Envision plate reader at 620 nM. Calculation percentage inhibition by using equation {% inhibition=100×[I−(X−MIN)/(MAX−MIN)]} where X equals to the well signal, Max equals signal of DMSO control and MIN equals signal of no cell control.

TABLE 2
Cellular IC₅₀ values of the compounds
of this invention against CD73

| Example No. | IC₅₀ (μM) |
|---|---|
| 1 | 0.074 |
| 2 | 0.156 |
| 3 | 0.027 |
| 4 | 0.107 |

### Example 7: CD73 LC/MS Assay

The purpose of this assay is to identify and characterize inhibitors of CD73 enzymatic activity. Compound serial dilution (1:3) was prepared with Echo 555 liquid handler (Labcyte) into the corresponding wells of a 384-well plate. 12.5 μL of enzyme working solution (containing recombinant CD73 protein, 10 mM Tris pH 7.5, 100 mM NaCl, 0.01% BSA, 0.2 mM Octyl glucoside) was added to the assay plate and incubated with compounds for 15 minutes at room temperature. After adding 15 μL of AMP working solution (containing 10 mM Tris pH 7.5, 100 mM NaCl, 0.01% BSA, 0.2 mM Octyl glucoside, with or without NaH₂PO₄), the assay was incubated for 10 min at room temperature. The reaction was stopped by adding 75 μL of stop solution (5% TCA in H₂O containing 250 nM 13C5-adenosine) to each well for 10 min incubation. After centrifugation, 75 μL of the mixture was transferred to a new 384-well plate for LC/MS analysis.

Samples from the 384-well plates were loaded onto an autosampler deck, then injected with ADDA-LC-MS/MS. The aqueous mobile phase is 0.1% formic acid in water. The organic mobile phase is 0.1% formic acid in acetonitrile. Flow rate is maintained at 0.8 mL/minute using Shimadzu pumps. The column is ACE 5 Phenyl, 50×2.1 mm. The analysis was performed on a SCIEX triple quadrupole mass spectrometer operating in positive ion mode. The effluent from the HPLC column was directly introduced into the electrospray ionization (ESI). Multiple reaction monitoring (MRM) is used to determine analyte and internal standard (IS) responses. The MRM for adenosine is 268.1/136.1, for 13C5-Adenosine (IS) is 273.2/136.2. The data is calculated using the peak area ratio (PAR) semi-quantitative method.

### Example 8: T Cell Proliferation Assay

The purpose of this assay is to characterize the potency of inhibitors of CD73 in rescuing adenosine-mediated inhibition of T cell proliferation. CD4+ or CD8+ T cells were isolated from peripheral blood mononuclear cells (PBMCs, HemaCare) by immunomagnetic negative selection using EasySep™ Isolation Kit (STEMCELL Technologies) following the supplier's protocol. CD4+ or CD8+ T cells were pelleted by centrifugation at 300 gravitational force (g) for 10 minutes at room temperature and re-suspended in PBS. CellTrace™ Violet staining solution (Invitrogen) was added at 1:2,000 and incubated at 37° C. for 20 minutes, protected from light. Complete culture medium [RPMI-1640 (Gibco), 10% Fetal Bovine Serum (Gibco), 2 mM GlutaMAX (Gibco) and 1 mM Sodium Pyruvate (Gibco), 100 U/mL Penicillin-Streptomycin (Gibco) and MEM non-essential amino acids (NEAA) cell culture supplement (1:100, Gibco)] was then added, mixed, and incubated at 37° C. for 5 minutes. Cells were then pelleted by centrifugation at 300 g for 10 minutes at room temperature and re-suspended in fresh, pre-warmed complete culture medium. 50 μL of cells were seeded per well in 96 well u-bottom plates. 50 μL of CD3/CD28 beads-containing medium were added into each well and incubated overnight at 37° C. in a 5% CO₂ incubator. 50 μL of media containing compounds was added into cells. 50 μL of media containing AMP and EHNA hydrochloride (Sigma-Aldrich) was added into cells at 200 μM and 5 μM final concentration, respectively. Cells were incubated for 72 hours at 37° C. in a 5% CO₂ incubator. 200 μL of PBS was then added to each well and cells were centrifuged at 300 g. 4° C. for 10 minutes. The supernatant was discarded. 50 μL of Human TruStain FcX™ (Fc Receptor Blocking Solution, BioLegend) diluted 1:100 in PBS was added to each well, mixed gently and incubated for 20 minutes at 4° C. 50 μL of staining solution (BioLegend) was added to each well, mixed gently and incubated at 4° C. for 30 minutes. Cells were centrifuged at 300 g, 4° C. for 10 minutes and the supernatant was discarded. The cell pellets were washed with 250 μL of cell staining buffer and centrifuged at 300 g. 4° C. for 10 minutes. The supernatant was discarded and cells were re-suspended in 60 μL of cell staining buffer and analyzed on a flow cytometer.

### Example 9: T Cell Cytokine Release Function Assay

The purpose of this assay is to characterize the potency of inhibitors of CD73 in rescuing adenosine-mediated inhibition of T cell cytokine release function. CD4+ or CD8+ T cells were isolated from peripheral blood mononuclear cells (PBMCs) by immunomagnetic negative selection using EasySep™ Isolation Kit (STEMCELL Technologies) following the supplier's protocol. CD4+ or CD8+ T cells were then pelleted the cells by centrifugation at 300 g for 10 minutes at room temperature and re-suspended in fresh, pre-warmed complete culture medium. 50 μL of cells was seeded per well in 96 well u-bottom plates. 50 μL of CD3/CD28 beads-containing medium were added into each well and incubated overnight at 37° C. in a 5% CO₂ incubator. 50 μL of media containing compounds was added into cells. 50 μL of media containing AMP and EHNA hydrochloride (Sigma-Aldrich) was added into cells at 200 UM and 5 μM final concentration, respectively. Cells were incubated for 72 hours at 37° C. in a 5% CO₂ incubator. 50 μL of supernatant was collected to determine levels of IL2 and IFN gamma using ELISA-MSD kit (Meso Scale Discovery).

1. A compound of formula (I), 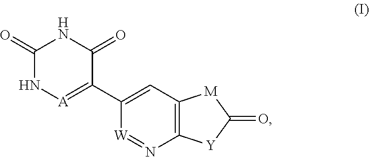
wherein
M is NR¹, wherein R¹ is C₁₋₆alkyl, C₃₋₇cycloalkyl, C₃₋₇cycloalkylC₁₋₆alkyl, or -L¹-R²; wherein L¹ is C₁₋₆alkylene, C₃₋₇cycloalkylene, heterocyclylene or heteroarylene; R² is optionally substituted phenyl, heteroaryl or benzoyl;
Y is NR³, wherein R³ is H, C₁₋₆alkyl, aryl, heteroaryl, C₃₋₇cycloalkyl or C₃₋₇cycloalkylC₁₋₆alkyl;
A is CH or N;
W is CH or N;
or a pharmaceutically acceptable salt thereof.

2. A compound according to claim 1, wherein R¹ is C₁₋₆alkyl or -L¹-R²; wherein L¹ is C₁₋₆alkylene; R² is phenyl.

3. A compound according to claim 1 or 2, wherein R¹ is methyl, isopropyl or benzyl.

4. A compound according to any one of claims 1-3, wherein R³ is H or C₁₋₆alkyl.

5. A compound according to any one of claims 1-4, wherein R³ is H or methyl.

6. A compound according to any one of claims 1-5, wherein A is CH.

7. A compound according to any one of claims 1-6, wherein W is N.

8. A compound according to claim 1, wherein
M is NR¹, wherein R¹ is C₁₋₆alkyl or -L¹-R²; wherein L¹ is C₁₋₆alkylene; R² is phenyl;
Y is NR³, wherein R³ is H or C₁₋₆alkyl;
A is CH;
W is N;
or a pharmaceutically acceptable salt thereof.

9. A compound according to claim 8, wherein
M is NR¹, wherein R¹ is methyl, isopropyl or benzyl;
Y is NR³, wherein R³ is H or methyl;
A is CH;
W is N;
or a pharmaceutically acceptable salt thereof.

10. A compound selected from:
5-(5-methyl-6-oxo-7H-imidazo[4,5-c]pyridazin-3-yl)-1H-pyrimidine-2,4-dione;
5-(5,7-dimethyl-6-oxo-imidazo[4,5-c]pyridazin-3-yl)-1H-pyrimidine-2,4-dione;
5-(5-isopropyl-7-methyl-6-oxo-imidazo[4,5-c]pyridazin-3-yl)-1H-pyrimidine-2,4-dione; and
5-(5-benzyl-7-methyl-6-oxo-imidazo[4,5-c]pyridazin-3-yl)-1H-pyrimidine-2,4-dione;
or a pharmaceutically acceptable salt thereof.

11. A process for the preparation of a compound according to any one of claims 1 to 10 comprising any of the following steps:
a) Deprotection of compound of formula (VII), 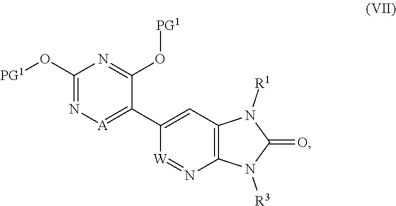
with acid or through hydrogenation to afford the compound of formula (VIII), 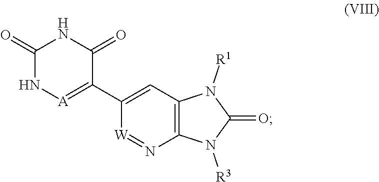
wherein PG¹ is methyl, tert-butyl, TBS, ethoxymethyl, or benzyl. R¹, R³, A and W are defined as in any one of claims 1 to 9; the acid in step a) is HCl.

12. A compound or pharmaceutically acceptable salt according to any one of claims 1 to 10 for use as therapeutically active substance.

13. A pharmaceutical composition comprising a compound in accordance with any one of claims 1 to 10 and a pharmaceutically acceptable excipient.

14. The use of a compound according to any one of claims 1 to 10 for treating cancers.

15. The use claim 14, wherein the cancer is pancreatic cancer, colorectal cancer, gastric cancer, esophageal cancer, liver cancer, lung cancer, breast cancer, ovarian cancer, prostate cancer or melanoma.

16. The use of a compound according to any one of claims 1 to 10 for inhibiting CD73.

17. The use of a compound according to any one of claims 1 to 10 for the preparation of a medicament for the treatment or prophylaxis of cancers, wherein the cancer is pancreatic cancer, colorectal cancer, gastric cancer, esophageal cancer, head and neck cancer, liver cancer, lung cancer, breast cancer, ovarian cancer, prostate cancer, melanoma, multiple myeloma, acute myeloid leukemia, or acute and chronic lymphoblastic leukemia.

18. The use of a compound according to any one of claims 1 to 10 for the preparation of a medicament as a CD73 inhibitor.

19. A compound or pharmaceutically acceptable salt according to any one of claims 1 to 10, when manufactured according to a process of claim 11.

20. The invention as hereinbefore described.
